(19) United States
(12) Patent Application Publication
(10) Pub. No.: US 2010/0022562 A1
(43) Pub. Date: Jan. 28, 2010

(54) Use of Plant Growth Regulators to Reduce Abscisic Acid Related Plant Leaf Yellowing

(76) Inventors: Xiaozhong Liu, Vernon Hills, IL (US); Derek D. Woolard, Zion, IL (US); Peter D. Petracek, Grayslake, IL (US)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/508,155

(22) Filed: Jul. 23, 2009

Related U.S. Application Data
(60) Provisional application No. 61/083,204, filed on Jul. 24, 2008.

(51) Int. Cl. A61K 31/52; A61K 31/19; A61K 31/44

(52) U.S. Cl. 514/263.4; 514/568; 514/357

(57) ABSTRACT

The present invention describes methods for maintaining ABA-induced drought tolerance while reducing ABA-induced leaf yellowing through the combination of ABA with selected plant growth regulators. The present invention also describes a method of using selected ABA analogs to reduce water use with minimal leaf yellowing.

### FIELD OF THE INVENTION

The present invention relates to methods of using certain plant growth regulators to selectively counteract ABA-induced leaf yellowing while not reducing ABA-induced drought tolerance. The present invention also relates to methods of using selected ABA analogs to reduce water use with minimal leaf yellowing.

### BACKGROUND OF THE INVENTION

Abscisic acid (ABA; S-abscisic acid, S-ABA) is a naturally-occurring plant hormone found in all higher plants (Cutler and Krochko. 1999. Trends in Plant Science. 4: 472-478.; Finkelstein and Rock. 2002. The Arabidopsis Book. ASPB, Monona, Md., 1-52). ABA is involved in many major events of plant growth and development including dormancy, germination, bud break, flowering, fruit set, growth and development, stress tolerance, ripening, abscission and senescence. ABA also plays an important role in plant tolerance to environmental stresses such as drought, cold and excessive salinity.

One key role of ABA in regulating physiological responses of plants is to act as a signal of reduced water availability to reduce water loss, inhibit growth, and induce adaptive responses. These functions are related in part to ABA-induced stomatal closure (Raschke and Hedrich 1985, Planta, 163: 105-118). When drought occurs, ABA synthesis increases. ABA accumulates in the plant leaves, induces stomata closure, reduces water use, and thus increases drought tolerance. Exogenous application of ABA can also be used to improve drought tolerance in most plants.

However, ABA may also induce undesirable effects such as leaf senescence and abscission in some plants. Geranium cuttings treated with ABA cause leaf yellowing (Mutui et al., 2005, J. Hort. Sci. Biotechnol. 80: 453-550). ABA-induced leaf yellowing has been observed in other ornamental plants including Agapanthus, Alyssum, Calibrachoa, Gazania, Lobelia, Pansy, Poinsettia, Rose and Vinca. This undesirable effect limits potential commercialization of ABA for these ornamental plants. Approaches for selectively reducing ABA-induced leaf yellowing while maintaining ABA-induced drought tolerance have not been reported. Commercialization of ABA or related compounds on plants like Pansy requires the discovery of ways to selectively achieve the desired treatment effects such as transpiration inhibition while minimizing the undesired treatment effect like leaf yellowing.

Cytokinins are known to delay plant leaf senescence and maintain leaf greenness (Biddington and Thomas, 1978. Physiol. Plant. 42: 369-3741; Funnel and Heins, 1998, HortScience. 33: 1036-1037; Reid, 2002, U.S. Pat. No. 6,455,466 B1). However, Blackman and Davies (1984. Ann. Bot. 54: 121-123) reported that the adenine-based cytokinin benzyladenine (6-BA; 6BA; BA) reverses ABA-induced closure of stomata of young maize leaves. These results suggest that cytokinins may reduce ABA-induced drought tolerance of plant species. The use of combinations of ABA and either adenine-based cytokinins such as 6-BA or urea-based cytokinins such as forchlorfenuron (CPPU) for selectively reducing ABA-induced leaf yellowing while maintaining ABA-induced drought tolerance has not been reported.

Ethylene inhibitors such as the synthesis inhibitor aminoethoxyvinylglycine (AVG) and the action inhibitor 1-methylcyclopropene (MCP) may prevent ethylene-related leaf senescence (Bardella et al., 2007, US 2007/0265166 A1). However, the use of combinations of ABA and ethylene inhibitors for selectively reducing ABA-induced leaf yellowing while maintaining ABA-induced drought tolerance has not been reported.

Gibberellins such as gibberellin A3 (GA3; gibberellic acid) and gibberellin A4/gibberellin A7 (GA₄₊₇; GA4/GA7; GA4/7) may prevent leaf senescence (Han, 1997, J. Amer. Soc. Hort. Sci. 122: 869-872; Han, 1997, J. Amer. Soc. Hort. Sci. 122: 869-872). However, the use of combinations of ABA and ethylene inhibitors for selectively reducing ABA-induced leaf yellowing while maintaining ABA-induced drought tolerance has not been reported.

Selected ABA analogs have been shown to effectively reduce ABA-related germination inhibition (Abrams and Gusta, 1993, U.S. Pat. No. 5,201,931; Wilen, et al., 1993, Plant Physiol. 101: 469-476). However, the use of combinations of ABA and ABA analogs for selectively reducing ABA-induced leaf yellowing while maintaining ABA-induced drought tolerance has not been reported.

Selected ABA analogs have been shown to effectively produce an ABA-like effect in reducing water use (Abrams et al. 1999, U.S. Pat. No. 6,004,905). However, the use of ABA analogs to reduce water use without inducing leaf yellowing has not been reported.

### SUMMARY OF THE INVENTION

The present invention is directed to the use of plant growth regulators to reduce abscisic acid (ABA; S-abscisic acid, S-ABA) induced leaf yellowing in certain ABA sensitive species such as Pansy without reducing ABA improved ornamental plant drought tolerance.

The present invention is also directed to the incorporation of an effective amount of a cytokinin into an ABA containing composition in order to decrease ABA plant leaf yellowing while retaining drought tolerance.

Presently preferred cytokinins include BA and CPPU.

The present invention is also directed to the incorporation of an effective amount of an ethylene inhibitor into an ABA containing composition in order to decrease ABA plant leaf yellowing while retaining drought tolerance.

Presently preferred ethylene inhibitors include MCP and AVG.

The present invention is also directed to the incorporation of an effective amount of a gibberellin into an ABA containing composition in order to decrease ABA plant leaf yellowing while retaining drought tolerance.

Presently preferred gibberellins include GA4/GA7 and GA3.

The present invention is also directed to the incorporation of an effective amount of the ABA analog PBI-51 (Abrams and Gusta, 1993, U.S. Pat. No. 5,201,931) into an ABA containing composition in order to decrease ABA plant leaf yellowing while retaining drought tolerance.

The present invention is also directed to the use of ABA analogs instead of ABA to induce drought tolerance with minimal induction of leaf yellowing. This is accomplished by applying said end-use solution composition directly to plants by spraying or drenching.

Presently preferred ABA analogs and derivatives include PBI-429 (8′ acetylene-ABA methyl ester) and PBI-524 (8′ acetylene-ABA, acid; Abrams et al. 1999, U.S. Pat. No. 6,004,905).

### DETAILED DESCRIPTION OF THE INVENTION

The applied concentration of ABA can vary widely depending on the water volume applied to plants as well as other factors such as the plant age and size, and plant sensitivity to ABA, but is generally in the range of about 1 ppm to about 10,000 ppm, preferably from about 10 to about 1000 ppm.

It is also contemplated that salts of ABA may be utilized in accordance with the present invention.

As used herein, the term “salt” refers to the water-soluble salts of ABA. Representative such salts include inorganic salts such as the ammonium, lithium, sodium, calcium, potassium and magnesium salts and organic amine salts such as the triethanolamine, dimethylethanolamine and ethanolamine salts.

Cytokinins useful in the present invention include adenine-type cytokinins such as 6-benzylaminopurine (benzyladenine; 6-BA; 6BA; BA), kinetin, or zeatin and phenylurea-type cytokinin such as N₁-(2-chloro-4-pyridyl)-N₃-phenylurea (forchlorfenuron; CPPU) or thidiazuron (TDZ).

Ethylene inhibitors useful in the present invention include the ethylene synthesis inhibitor aminoethoxyvinylglycine (AVG) and the action inhibitor 1-methylcyclopropene (1-MCP).

Gibberellins useful in the present invention include gibberellin A3 (GA3; gibberellic acid) and gibberellin A4/gibberellin A7 (GA₄₊₇; GA₄/GA₇; GA_{4/7}).

ABA analogs that selectively antagonize ABA activity that are useful in the present invention include PBI-51 (Abrams and Gusta, 1993, U.S. Pat. No. 5,201,931; Wilen, et al., 1993, Plant Physiol. 101: 469-476):

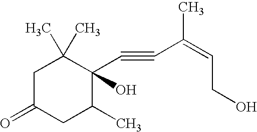

Presently preferred ABA analogs and derivatives useful in the present invention include PBI-429, PBI-524, PBI-696 and PBI-702.

For the purposes of this Application, abscisic acid analogs are defined by Structures 1, 2 and 3, wherein for Structure 1:

the bond at the 2-position of the side chain is a cis- or trans- double bond,

the bond at the 4-position of the side chain is a trans- double bond or a triple bond,

the stereochemistry of the hydroxyl group substituent on the ring is S-, R- or an R,S-mixture,

the stereochemistry of the R₁ group is in a cis- relationship to the hydroxyl group substituent on the ring,
R₁ is ethynyl, ethenyl, cyclopropyl or trifluoromethyl, and
R₂ is hydrogen or lower alkyl

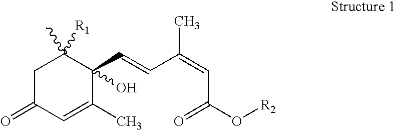

wherein lower alkyl is defined as an alkyl group containing 1 to 4 carbon atoms in a straight or branched chain, which may comprise zero or one ring or double bond when 3 or more carbon atoms are present.

For PBI-429, R₁ is ethynyl and R₂ is a methyl group.

For PBI-524, R₁ is ethynyl and R₂ is hydrogen.

For PBI-696, R₁ is cyclopropyl and R₂ is a methyl group.

### For Structure 2:

the bond at the 2-position of the side chain is a cis- or trans- double bond,

the bond at the 4-position of the side chain is a triple bond,

the stereochemistry of the hydroxyl group substituent on ring structure is S-, R- or an R,S-mixture,

R₁ is hydrogen or lower alkyl

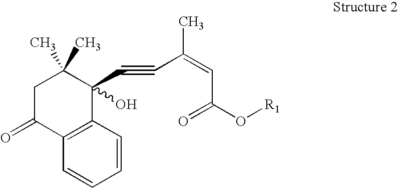

wherein lower alkyl is defined as an alkyl group containing 1 to 4 carbon atoms in a straight or branched chain, which may comprise zero or one ring or double bond Ahen 3 or more carbon atoms are present.

For PBI-702, R₁ is a methyl group.

### For Structure 3:

the bond at the 2-position of the side chain is a cis- or trans- double bond, the bond at the 4-position of the side chain is a trans-double bond,

the stereochemistry of the hydroxyl group substituent on the ring structure is S-, R- or an R,S-mixture,

R₁ is hydrogen or lower alkyl

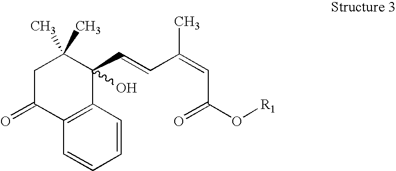

wherein lower alkyl is defined as an alkyl group containing 1 to 4 carbon atoms in a straight or branched chain, which may comprise zero or one ring or double bond when 3 or more carbon atoms are present.

For PBI-488, R₁ is a methyl group.

The invention is demonstrated by, but is not limited by, the following representative examples.

### EXAMPLES

All studies were conducted in the greenhouse at the research farm of Valent BioSciences Corporation (Long Grove, Ill.). Pansy plants were obtained either from local retailers as mature plants, or plugs from wholesale nurseries. Plugs of Pansy plants were transplanted into an 18-cell flat filled with Promix BX (available from Premier Horticulture Inc. Quakertown, Pa.) and grown for about 30 days prior to treatment. During growing periods, plants received daily irrigation and weekly fertilizer (1 g/L all purpose fertilizer 20-20-20, The Scotts Company, Marysville, Ohio).

Chemical solutions were prepared with distilled water. Abscisic acid (S-ABA; ABA; S-(+)-abscisic acid; +-ABA, (+)-(S)-cis,trans-abscisic acid,(+)-(S)-cis,trans-ABA; S-ABA; (S)-5-(1-hydroxy-2,6,6,-trimethyl-4-oxo-2-cyclohexen-1-yl)-3-methyl-(2Z,4E)-pentadienoic acid; CAS no. 21293-29-8, 10% active ingredient), N6-benzyladenine (benzyladenine, 6BA, BA), forchlorfenuron (CPPU), aminoethoxyvinylglycine (AVG), gibberellic acid (GA3), gibberellin A₄₊₇ mixture (GA₄₊₇) were obtained from Valent BioSciences Corporation (Libertyville, Ill.). Ethyl-Bloc with active ingredient 1-methylcyclopropene (MCP) was obtained from Floralife®, Inc. (Walterboro, S.C.).

ABA analogs, 8′ acetylene-ABA, acid (PBI-524), 8′ acetylene-ABA methyl ester (PBI-429), 8′ cyclopropane ester (PBI-696), tetralone, first carbon tail acetylene, ester (PBI-702), tetralone, ester (PBI-488) and the reported ABA antagonist PBI-51 (Abrams and Gusta, 1993, U.S. Pat. No. 5,201,931; Wilen, et al., 1993, Plant Physiol. 101: 469-476) were synthesized by Plant Biotechnology Institute, National Research Council of Canada (Saskatoon, Saskatchewan, Canada).

Uniform plants were selected for the study. Prior to chemical treatment, plants were saturated with water and then drained for about two hours. A total of 20 mL chemical solution, which is equivalent to about 10% of the cell volume, was applied to each plant with 3 mL solution foliar applied to canopy and 17 mL solution drench applied to root zone. Unless specified, watering of plants was stopped after chemical treatment.

After chemical treatment, plants were arranged in a randomized complete block experimental design. The plants were rated daily for the extent of wilting on a scale from 1 (no wilting) to 4 (complete wilting) to generated a sales index rating. A rating of 2.5 was the point at which a plant was determined to be unmarketable and the previous day was recorded as the shelf life of that plant in days. Yellow leaf number was counted at 3 days after chemical treatment. Leaf transpiration rate was measured after treatment using a LI-1600 Steady State Porometer (LI-Cor, Lincoln, Nebr.). The transpiration rate of each treatment was calculated as the percentage of that of control at each day to reduce day-to-day variation caused by changes of environmental condition such as light intensity, humidity, and temperature.

In Examples 1 and 2, selected analogs of ABA are shown to extend shelf life under drought stress with less leaf yellowing than ABA.

In Examples 3 to 14, selected chemicals (PBI-5 1, BA, CPPU, trinexapac, AVG, or MCP) are shown to reduce ABA or ABA analog induced leaf yellowing without reducing shelf life under drought stress.

In total, these examples show that the ABA related treatment effects of transpiration reduction and leaf yellowing are separable.

### Example 1

Individual pansy plants were treated with 20 mL of treatment solution (sprayed 3 mL and drenched 17 mL). Treatment solutions contained: 1, 3, 10 or 30 mg ABA: 0.1, 0.3, 1 or 3 mg PBI-429; or water. The dose range of PBI-429 was used at one-tenth of ABA dose based on the preliminary results on drought tolerance. Irrigation was withheld until all the plants wilted. Plants were individually rated daily to determine the sales index value. Yellow leaf numbers were counted 3 days after treatment.

Both ABA and PBI-429 extended Pansy shelf life under drought condition in a dose dependent manner (Table 1). Pansy shelf life for the 1 mg or 3 mg PBI-429 treatment was similar to 10 mg or 30 mg ABA treatment, respectively.

ABA and PBI-429 also increased yellow leaf number in a dose response manner. Surprisingly, the number of yellow leaves on PBI-429 treated plants was similar to plants treated with same dose of ABA. Thus, PBI-429 achieved the same level of drought tolerance as ABA, but with substantially less leaf yellowing.

TABLE 1
Effect of ABA and ABA analog PBI-429 on Pansy
(Variety: Matrix Orange) shelf life and yellow leaf
number under drought condition.

| Treatment | Shelf life (days) | Yellow leaf number |
|---|---|---|
| Control | 5.2 | 2 |
| 1 mg ABA | 5.0 | 8 |
| 3 mg ABA | 5.8 | 11 |
| 10 mg ABA | 7.0 | 14 |
| 30 mg ABA | 8.0 | 18 |
| 0.1 mg PBI-429 | 5.5 | 3 |
| 0.3 mg PBI-429 | 5.8 | 5 |
| 1 mg PBI-429 | 7.0 | 7 |
| 3 mg PBI-429 | 8.3 | 10 |

### Example 2

Five ABA analogs (PBI-429, PBI-524, PBI-696, PBI-702, and PBI-488) were evaluated for their ability to increase Pansy drought tolerance and their effect on leaf yellowing. Pansy plants (variety Matrix Orange) were treated with 0.3 mg or 3 mg of each ABA analog and compared to 3 mg, 10 mg, or 30 mg ABA.

At the higher dose (3 mg), shelf lives of PBI-429 and PBI-524 treated Pansy plants were similar to 30 mg ABA treated plants (Table 2). The shelf life of plants treated with 3 mg PBI-696 was between the shelf lives of plants treated with 10 and 30 mg ABA. The shelf lives of plants treated with PBI-702 and PBI-488 were similar to plants treated with 10 mg ABA. At the lower dose (0.3 mg), the shelf life of ABA analog treated plants was similar to 3 mg ABA treated plants.

Although Pansy shelf life extension differed among the tested ABA analogs, surprisingly, yellow leaf number caused by different ABA analogs was similar. Yellow leaf number caused by 0.3 mg or 3 mg ABA analog tended to be no more than the yellow leaf number caused by respective doses of ABA. These results show that treatment with selected ABA analogs can achieve shelf life extension with proportionally less leaf yellowing than treatment with ABA.

TABLE 2
Effect of ABA and ABA analogs on Pansy
(Variety: Matrix Orange) shelf life and yellow leaf
number under drought condition.

| Treatment | Shelf life (days) | Yellow leaf number |
|---|---|---|
| Control | 3.8 | 9.0 |
| 3 mg ABA | 5.2 | 12.0 |
| 10 mg ABA | 6.7 | 15.2 |
| 30 mg ABA | 9.2 | 21.3 |
| 0.3 mg PBI-429 | 5.8 | 9.0 |
| 3 mg PBI-429 | 9.5 | 12.2 |
| 0.3 mg PBI-524 | 6.5 | 11.0 |
| 3 mg PBI-524 | 8.8 | 11.2 |
| 0.3 mg PBI-696 | 5.3 | 9.0 |
| 3 mg PBI-696 | 7.8 | 12.3 |
| 0.3 mg PBI-702 | 4.7 | 10.8 |
| 3 mg PBI-702 | 7.0 | 11.0 |
| 0.3 mg PBI-488 | 5.3 | 9.2 |
| 3 mg PBI-488 | 7.0 | 11.5 |

### Example 3

A reported ABA antagonist PBI-51 was used to test its role alone and in combination with ABA to improve ABA drought tolerance and reduce Pansy leaf yellowing. As the results show in Table 3, plants treated with 3 mg or 30 mg PBI-51 had similar shelf life and yellow leaf number. However, for plants treated with the combination of 30 mg ABA with 3 or 30 mg PBI-51, the yellow leaf number decreased compared to 30 mg ABA alone. Surprisingly, Pansy plants treated with ABA and PBI-51 combination had a similar shelf life compared to plants treated with ABA alone. These results show that PBI-51 selectively reduces ABA-induced yellowing without decreasing ABA-extension of shelf life.

TABLE 3
Effect of ABA analog PBI-51 on ABA related Pansy
(Variety: Delta Premium Pure Golden Yellow) leaf
yellowing under drought condition.

| Treatment | Shelf Life (days) | Yellow leaf number |
|---|---|---|
| Control | 4.3 | 7.2 |
| 3 mg ABA | 5.3 | 11.0 |
| 10 mg ABA | 6.5 | 14.7 |
| 30 mg ABA | 8.0 | 21.0 |
| 3 mg PBI-51 | 5.0 | 7.5 |
| 30 mg PBI-51 | 5.0 | 8.0 |
| 30 mg ABA + 3 mg PBI-51 | 8.2 | 15.3 |
| 30 mg ABA + 30 mg PBI-51 | 8.3 | 14.7 |

Similar results were found when the same treatments were applied to Pansy plants in an advanced seedling stage (1 month old). PBI-51 could be used to reduce Pansy leaf yellowing caused by ABA while not affecting Pansy shelf life (Table 4).

TABLE 4
Effect of ABA analog PBI-51 on ABA related Pansy
(Variety: Delta Premium Pure Golden Yellow) leaf
yellowing under drought condition.

| Treatment | Shelf Life (days) | Yellow leaf number |
|---|---|---|
| Control | 3.2 | 9.5 |
| 3 mg ABA | 4.2 | 13.8 |
| 10 mg ABA | 5.7 | 19.0 |
| 30 mg ABA | 7.7 | 28.2 |
| 3 mg PBI-51 | 3.7 | 8.2 |
| 30 mg PBI-51 | 3.5 | 8.8 |
| 30 mg ABA + 3 mg PBI-51 | 7.5 | 16.8 |
| 30 mg ABA + 30 mg PBI-51 | 8.2 | 19.8 |

### Example 4

The adenine-based cytokinin benzyladenine (BA; 6-BA) was combined with ABA to treated Pansy plants. Pansy plants treated with the BA and ABA combinations had fewer yellow leaves than plants treated with ABA alone at the same ABA level (Table 5). Plants treated with a high dose of BA (2 mg) had fewer yellow leaves than plants treated with a low dose of BA (0.2 mg). Although it would be expected that BA would reduce the effect of ABA on shelf life, the pansy shelf life for plants treated with ABA and BA combination was not different from the same dose of ABA treated plants. This shows that BA selectivity reduces ABA induced leaf yellowing without substantially reducing ABA extension of shelf life.

TABLE 5
Effect of BA on ABA related Pansy (Variety: Matrix
Yellow) shelf life and leaf yellowing.

| Treatment | Shelf life (days) | Yellow leaf number |
|---|---|---|
| Control | 3.7 | 3.8 |
| 0.3 mg ABA | 4.8 | 5.3 |
| 3 mg ABA | 5.7 | 7.5 |
| 30 mg ABA | 10.2 | 25.2 |
| 0.2 mg BA | 3.8 | 1.8 |
| 0.3 mg ABA + 0.2 mg BA | 4.7 | 3.2 |
| 3 mg ABA + 0.2 mg BA | 5.5 | 5.7 |
| 30 mg ABA + 0.2 mg BA | 10.2 | 10.5 |
| 2 mg BA | 3.8 | 1.5 |
| 0.3 mg ABA + 2 mg BA | 4.8 | 1.8 |
| 3 mg ABA + 2 mg BA | 6.5 | 2.2 |
| 30 mg ABA + 2 mg BA | 9.3 | 7.7 |

### Example 5

The urea-based cytokinin CPPU was also combined with ABA to treated Pansy plants. Similar to BA, CPPU also greatly decreased but did not eliminate the Pansy yellow leaf number. CPPU also did not affect Pansy shelf life (Table 6).

TABLE 6
Effect of CPPU on ABA related Pansy (Variety: Matrix
Yellow) shelf life and leaf yellowing.

| | | Shelf life | |
|---|---|---|---|
| Treatment | | (days) | Yellow leaf number |
| | Control | 2.8 | 7.8 |
| 0.3 | mg ABA | 4.8 | 7.8 |
| 3 | mg ABA | 6.0 | 10.2 |
| 30 | mg ABA | 10.8 | 17.3 |
| 0.02 | mg CPPU | 3.2 | 7.7 |
| 0.3 | mg ABA + 0.02 mg CPPU | 4.3 | 7.7 |
| 3 | mg ABA + 0.02 mg CPPU | 6.0 | 8.7 |
| 30 | mg ABA + 0.02 mg CPPU | 11.5 | 9.2 |
| 0.2 | mg CPPU | 2.8 | 5.7 |
| 0.3 | mg ABA + 0.2 mg CPPU | 3.8 | 6.3 |
| 3 | mg ABA + 0.2 mg CPPU | 5.3 | 7.5 |
| 30 | mg ABA + 0.2 mg CPPU | 10.8 | 8.0 |

### Example 6

The effect of BA on ABA or ABA analog (PBI-429) induced Pansy leaf yellowing was tested with the variety Matrix Yellow. Matrix Yellow Pansy treated with 0.3 mg PBI-429 or 3 mg PBI-429 had the same shelf life as 3 mg ABA or 30 mg ABA-treated Pansy plants. However, PBI-429 treated Pansy plants had a much lower yellow leaf number than ABA treated Pansy plants. Pansy plants treated with the combination of BA with 30 mg ABA or 3 mg PBI-429 had similar shelf life as 30 mg ABA or 3 mg PBI-429 treated Pansy plants (Table 7). Pansy plants treated with the combination of BA with 30 mg ABA or 3 mg PBI-429 had a much lower yellow leaf number than 30 mg ABA or 3 mg PBI-429 treated Pansy plants. Pansy treated plants with 2 mg BA and 3 mg PBI-429 had a lower yellow leaf number than Pansy treated plants with 2 mg BA and 30 mg PBI-ABA.

TABLE 7
Effect of BA on ABA and PBI-429 related Pansy (variety:
Matrix Yellow) shelf life and yellow leaf number under
drought condition.

| Treatment | Shelf Life (days) | Yellow leaf number |
|---|---|---|
| Control | 4.2 | 9.0 |
| 3 mg ABA | 7.7 | 11.3 |
| 30 mg ABA | 9.3 | 21.0 |
| 0.3 mg PBI-429 | 7.5 | 9.7 |
| 3 mg PBI-429 | 8.8 | 13.2 |
| 2 mg BA | 3.5 | 7.8 |
| 30 mg ABA + 2 mg BA | 9.3 | 15.2 |
| 3 mg PBI-429 + 2 mg BA | 9.3 | 11.0 |

Similar results were found in two different Pansy varieties, Clear Sky Yellow (Table 8) and Crown Azure Blue (Table 9). Results demonstrated that BA reduced ABA or ABA analog induced leaf yellowing without affecting its shelf life.

TABLE 8
Effect of BA on ABA and PBI-429 related Pansy (variety: Clear Sky
Yellow) shelf life and yellow leaf number under drought condition.

| Treatment | Shelf Life (days) | Yellow leaf number |
|---|---|---|
| Control | 4.7 | 0.5 |
| 3 mg ABA | 6.5 | 3.2 |
| 30 mg ABA | 7.8 | 9.2 |
| 0.3 mg PBI-429 | 6.5 | 2.8 |
| 3 mg PBI-429 | 8.7 | 4.8 |
| 2 mg BA | 3.8 | 0.0 |
| 30 mg ABA + 2 mg BA | 7.3 | 4.5 |
| 3 mg PBI-429 + 2 mg BA | 7.7 | 1.3 |

TABLE 9
Effect of BA on ABA and PBI-429 related Pansy (variety: Crown
Azure Blue) shelf life and yellow leaf number under drought condition.

| Treatment | Shelf Life (days) | Yellow leaf number |
|---|---|---|
| Control | 3.7 | 3.5 |
| 3 mg ABA | 5.2 | 5.7 |
| 30 mg ABA | 6.7 | 15.0 |
| 0.3 mg PBI-429 | 4.5 | 4.8 |
| 3 mg PBI-429 | 7.0 | 7.7 |
| 2 mg BA | 3.3 | 0.3 |
| 30 mg ABA + 2 mg BA | 6.3 | 5.7 |
| 3 mg PBI-429 + 2 mg BA | 6.2 | 6.0 |

### Example 7

ABA at 3 mg or 30 mg, 2 mg BA, 30 mg trinexepac-ethyl (TE), or their combinations were tested for their efficacy in increasing Pansy drought tolerance without increasing leaf yellowing. As results show in Table 10, the combination of 2 mg BA with 3 mg ABA or 30 mg ABA reduced Pansy yellow leaf number without affecting Pansy shelf life compared to Pansy plants treated with same dose of ABA alone. The combination of 30 mg TE with 3 mg ABA or 30 mg ABA extended Pansy shelf life compared with 3 mg ABA or numerically compared with 30 mg ABA. However, the combination of 30 mg TE with 3 mg ABA or 30 mg ABA did not affect the yellow leaf number. The combination of BA and TE with 3 mg ABA or 30 mg ABA reduced yellow leaf number as well as extended Pansy shelf life (3 mg ABA) or numerically (30 mg ABA).

TABLE 10
Effect of BA and trinexapac-ethyl (TE) on ABA related Pansy (Variety:
Matrix Yellow) shelf life and yellow leaf number under drought condition.

| | Shelf life | |
|---|---|---|
| Treatment | (days) | Yellow leaf number |
| Control | 2.7 | 8.2 |
| 3 mg ABA | 4.3 | 14.7 |
| 30 mg ABA | 8.0 | 21.2 |
| 2 mg BA | 2.3 | 7.2 |
| 3 mg ABA + 2 mg BA | 4.0 | 12.0 |
| 30 mg ABA + 2 mg BA | 7.5 | 14.3 |
| 30 mg TE | 2.5 | 8.7 |
| 3 mg ABA + 30 mg TE | 5.2 | 11.5 |
| 30 mg ABA + 30 mg TE | 8.5 | 13.7 |
| 2 mg BA + 30 mg TE | 2.8 | 7.5 |
| 3 mg ABA + 2 mg BA + 30 mg TE | 5.7 | 11.0 |
| 30 mg ABA + 2 mg BA + 30 mg TE | 8.7 | 13.3 |

### Example 8

In order to test the timing of BA application on ABA induced leaf yellowing, 2 mg BA was applied 1 day prior to, the same day as, or 1 day after a 30 mg ABA application. Results in Table 11 demonstrate that BA applied at any time reduced yellow leaf number. Plants treated earlier with BA had a lower number of yellow leaves. Pansy shelf life did not change when BA was applied at the same day as or 1 day after ABA treatment. When BA was applied 1 day prior to ABA application, Pansy plants had shorter shelf life.

TABLE 11
Pansy (Matrix Premium Rose) shelf life and yellow leaf number
as affected by ABA treatment in combination with different
timing of BA treatment.

| Treatment | Shelf life (day) | Yellow leaf number |
|---|---|---|
| Control | 4.0 | 2.0 |
| 30 mg ABA | 7.2 | 14.0 |
| 2 mg BA at −1 d | 3.8 | 0.7 |
| 30 mg ABA + 2 mg BA at −1 d | 6.5 | 8.7 |
| 2 mg BA at 0 d | 3.7 | 0.8 |
| 30 mg ABA + 2 mg BA at 0 d | 7.3 | 9.0 |
| 2 mg BA at +1 d | 4.0 | 0.8 |
| 30 mg ABA + 2 mg BA at +1 d | 7.3 | 9.7 |

No water was added 1 day after ABA treatment.
BA was applied 1 day prior to (at −1 day), same day (at 0 day), or 1 day after (at +1 day) ABA treatment.
Same amount of water was added when plants were not treated with BA.

In order to explore the mechanism of the ABA and BA combination effect on drought tolerance of Pansy plants, leaf transpiration was measured. BA alone tended to increase Pansy leaf transpiration compare to the control (Table 12). 30 mg ABA dramatically inhibited Pansy leaf transpiration. ABA inhibition of Pansy leaf transpiration was not affected by BA regardless of the time of application.

TABLE 12
Pansy (Matrix Premium Rose) leaf transpiration as affected by ABA
treatment in combination with different timing of BA treatment.

| | Transpiration rate (% of control) | | |
|---|---|---|---|
| | Days after ABA treatment | | |
| Treatment | 1 | 2 | 3 |
| Control | 100 | 100 | 100 |
| 30 mg ABA | 6 | 8 | 11 |
| 2 mg BA at −1 d | 115 | 104 | 109 |
| 30 mg ABA + 2 mg BA at −1 d | 4 | 9 | 14 |
| 2 mg BA at 0 d | 105 | 97 | 98 |
| 30 mg ABA + 2 mg BA at 0 d | 7 | 9 | 11 |
| 2 mg BA at +1 d | 96 | 101 | 107 |
| 30 mg ABA + 2 mg BA at +1 d | 5 | 7 | 16 |

No water was added 1 day after ABA treatment.
BA was applied 1 day prior to (at −1 day), same day (at 0 day), or 1 day after (at +1 day) ABA treatment.
Same amount of water was added when plants were not treated with BA.

### Example 9

Pansy plants were treated with 3 mg or 30 mg ABA alone or in combination with 2 mg BA. Plants were split into two regimes with daily water or no water. Plants that received daily watering survived through the experiment. Under no water (drought) conditions, ABA increased shelf life and also caused an increased number of yellow leaves (Table 13). The addition of BA to the ABA treatment solution reduced yellow leaf number without changing Pansy shelf life.

TABLE 13
Pansy (Matrix Premium Rose) shelf life and yellow leaf number as
affect by ABA and BA combination at no watered condition

| Water | | | Yellow leaf |
|---|---|---|---|
| Treatment | Chemical Treatment | Shelf Life (days) | number |
| No | Control | 3.8 | 1.7 |
| water | 3 mg ABA | 4.8 | 8.3 |
| | 30 mg ABA | 7.2 | 15.3 |
| | 2 mg BA | 4.0 | 0.8 |
| | 3 mg ABA + 2 mg BA | 4.8 | 5.2 |
| | 30 mg ABA + 2 mg BA | 7.0 | 8.2 |

Pansy leaf transpiration was measured. For plants receiving water (watered), BA did not affect Pansy leaf transpiration. However, both 3 mg and 30 mg ABA inhibited transpiration. ABA (3 mg) inhibited more than 50% transpiration within 5 days after treatment and was no longer effective at 10 days after treatment. ABA (30 mg) inhibited transpiration by more than 50% through 10 days after treatment and the effect disappeared by 15 days after treatment. The BA and ABA combination inhibited leaf transpiration similar to ABA alone (Table 14).

For plants not receiving water (no water), the transpiration rate of untreated Pansy plants decreased overtime, beginning at 2 days after treatment (Table 14). Thereafter leaves began wilting and eventually died. BA treatment showed a similar pattern as control plants. Pansy plants treated with ABA had a lower transpiration rate immediately after chemical treatments. The transpiration rate increased as the ABA effect diminished. Plants started wilting after the ABA effect on transpiration had sufficiently diminished.

TABLE 14
Pansy (Matrix Premium Rose) leaf transpiration rate as affect by ABA
and BA combination under watered and no watered condition.

| | | Transpiration rate (% of control) | | | | | | | |
|---|---|---|---|---|---|---|---|---|---|
| Water | Chemical | Days after treatment | | | | | | | |
| Treatment | Treatment | 1 | 2 | 3 | 4 | 5 | 7 | 10 | 15 |
| Watered | Control | 100 | 100 | 100 | 100 | 100 | 100 | 100 | 100 |
| | 3 mg ABA | 23 | 18 | 36 | 43 | 59 | 76 | 97 | 97 |
| | 30 mg ABA | 7 | 7 | 9 | 15 | 21 | 28 | 56 | 99 |
| | 2 mg BA | 109 | 102 | 101 | 101 | 99 | 98 | 101 | 98 |
| | 3 mg ABA + | 23 | 17 | 35 | 41 | 61 | 80 | 100 | 101 |
| | 2 mg BA | | | | | | | | |
| | 30 mg ABA + | 8 | 8 | 9 | 13 | 21 | 29 | 58 | 97 |
| | 2 mg BA | | | | | | | | |
| No water | Control | 100 | 69 | 57 | 38 | — | — | — | — |
| | 3 mg ABA | 22 | 15 | 34 | 35 | 27 | — | — | — |
| | 30 mg ABA | 7 | 8 | 8 | 15 | 21 | 27 | — | — |
| | 2 mg BA | 108 | 71 | 54 | 35 | — | — | — | — |
| | 3 mg ABA + | 21 | 15 | 34 | 34 | 31 | — | — | — |
| | 2 mg BA | | | | | | | | |
| | 30 mg ABA + | 7 | 9 | 6 | 16 | 21 | 26 | — | — |
| | 2 mg BA | | | | | | | | |

### Example 10

Under sufficient water conditions, Pansy plants survived during the experiment period so shelf life was not assessed. Under no water (drought) condition, ABA analog PBI-429 extended Pansy shelf life and caused leaf yellowing in a dose response manner. The combination of BA with PBI-429 reduced Pansy yellow leaf number, but did not affect Pansy shelf life (Table 15).

TABLE 15
Pansy (Whispers White) shelf life and yellow leaf number as affect by
ABA analog (PBI-429) and BA combination at no watered condition

| Water | | | Yellow leaf |
|---|---|---|---|
| Treatment | Chemical Treatment | Shelf Life (days) | number |
| No water | Control | 2.7 | 8.5 |
| | 0.3 mg PBI-429 | 3.8 | 12.3 |
| | 3 mg PBI-429 | 7.2 | 23.3 |
| | 0.2 mg BA | 2.5 | 7.8 |
| | 2 mg BA | 2.7 | 6.3 |
| | 0.3 mg PBI-429 + 0.2 mg | 4.7 | 9.0 |
| | BA | | |
| | 3 mg PBI-429 + 0.2 mg | 7.3 | 19.3 |
| | BA | | |
| | 0.3 mg PBI-429 + 2 mg | 4.5 | 9.3 |
| | BA | | |
| | 3 mg PBI-429 + 2 mg | 6.8 | 13.8 |
| | BA | | |

Under sufficient water conditions, PBI-429 at 0.3 mg or 3 mg inhibited Pansy leaf transpiration. The transpiration inhibition by 0.3 mg PBI-429 was greater than 50% through 3 days after treatment and substantially declined at 10 days after treatment. The transpiration inhibition by 3 mg PBI-429 was greater than 50% through 10 days after treatment. BA alone at 0.2 mg or 2 mg did not affect Pansy leaf transpiration. The Pansy leaf transpiration rate for plants treated by BA and PBI-429 combination was the same as the rate for Pansy plants treated with same rate of PBI-429 (Table 16).

Under the no water (drought) condition, the transpiration rate of the control plant leaf decreased, beginning at 2 days after treatment. Pansy leaves started wilting, beginning at 3 days after treatment (data not shown). The transpiration patterns of the 0.2 mg or 2 mg BA treated plants were similar to control plants. The transpiration rate of 0.3 mg or 3 mg PBI-429 treated plant leaves were maintained at low levels until plant wilted. The treated plants remained turgid longer than control plants. The transpiration rate of 3 mg PBI-429 treated plant leaves was lower than 0.3 mg PBI-429 treated plants. The plants treated with 3 mg PBI-429 remained turgid longer than plants treated with 0.3 mg PBI-429. The transpiration patterns of Pansy plants treated with BA and PBI-429 combinations were similar to plants treated with same dose of PBI-429 (Table 16).

TABLE 16
Pansy (Matrix Premium Rose) leaf transpiration rate as affect
by ABA analog (PBI-429) and BA combination under watered
and no water condition.

| | | Transpiration rate (% of control) | | | | | | |
|---|---|---|---|---|---|---|---|---|
| Water | | Days after treatment | | | | | | |
| Treatment | Chemical Treatment | 1 | 2 | 3 | 4 | 5 | 7 | 10 |
| Watered | Control | 100 | 100 | 100 | 100 | 100 | 100 | 100 |
| | 0.3 mg PBI-429 | 32 | 33 | 44 | 68 | 74 | 81 | 88 |
| | 3 mg PBI-429 | 20 | 15 | 29 | 28 | 32 | 44 | 46 |
| | 0.2 mg BA | 102 | 98 | 100 | 101 | 100 | 99 | 100 |
| | 2 mg BA | 99 | 97 | 98 | 97 | 101 | 97 | 102 |
| | 0.3 mg PBI-429 + 0.2 mg | 36 | 33 | 46 | 69 | 74 | 80 | 88 |
| | BA | | | | | | | |
| | 3 mg PBI-429 + 0.2 mg | 21 | 13 | 29 | 26 | 32 | 43 | 47 |
| | BA | | | | | | | |
| | 0.3 mg PBI-429 + 2 mg | 41 | 34 | 44 | 66 | 73 | 81 | 89 |
| | BA | | | | | | | |
| | 3 mg PBI-429 + 2 mg | 22 | 12 | 29 | 26 | 33 | 43 | 47 |
| | BA | | | | | | | |
| No water | Control | 100 | 51 | 7 | — | — | — | — |
| | 0.3 mg PBI-429 | 34 | 35 | 26 | — | — | — | — |
| | 3 mg PBI-429 | 23 | 14 | 9 | 10 | 18 | — | — |
| | 0.2 mg BA | 101 | 29 | 7 | — | — | — | — |
| | 2 mg BA | 101 | 34 | 14 | — | — | — | — |
| | 0.3 mg PBI-429 + 0.2 mg | 36 | 33 | 32 | — | — | — | — |
| | BA | | | | | | | |
| | 3 mg PBI-429 + 0.2 mg | 21 | 14 | 10 | 11 | 18 | — | — |
| | BA | | | | | | | |
| | 0.3 mg PBI-429 + 2 mg | 34 | 34 | 32 | — | — | — | — |
| | BA | | | | | | | |
| | 3 mg PBI-429 + 2 mg | 23 | 15 | 13 | 10 | 18 | — | — |
| | BA | | | | | | | |

### Example 11

The impact of aminoethoxyvinylglycine (AVG), an ethylene biosynthesis inhibitor, on ABA treatment of Pansy was examined. Pansy plants (Matrix Yellow) were treated with 2 or 20 mg AVG alone or in combination with 0.3, 3, or 30 mg ABA. The addition of 2 or 20 mg AVG to ABA did not affect the shelf life of Pansy plants compared to those plants treated with same dose of ABA (Table 17). The addition of 2 mg AVG to 3 or 30 mg ABA reduced Pansy yellow leaf number at 7 days after treatment compared to those plants treated with 3 or 30 mg ABA alone. The addition of 20 mg AVG to ABA increased the Pansy yellow leaf number compared to plants treated with same dose of ABA. This increase in yellow leaf number may be related to the phytotoxicity of high doses of AVG because 20 mg AVG alone also increased Pansy yellow leaf number compared to the control plants.

TABLE 17
Effect of AVG on ABA related Pansy (Matrix Yellow)
yellow leaf number and shelf life.

| | | Yellow leaf number | |
|---|---|---|---|
| | | Days after treatment | |
| Treatment | Shelf Life (days) | 3 | 7 |
| Control | 2.5 | 6.3 | 9.7 |
| 0.3 mg ABA | 3.2 | 7.0 | 12.0 |
| 3 mg ABA | 4.3 | 12.7 | 17.8 |
| 30 mg ABA | 8.3 | 19.3 | 28.5 |
| 2 mg AVG | 2.8 | 6.0 | 9.7 |
| 0.3 mg ABA + 2 mg AVG | 3.5 | 7.3 | 12.0 |
| 3 mg ABA + 2 mg AVG | 4.8 | 13.3 | 16.2 |
| 30 mg ABA + 2 mg AVG | 8.2 | 17.3 | 22.0 |
| 20 mg AVG | 3.2 | 6.3 | 11.3 |
| 0.3 mg ABA + 20 mg AVG | 4.0 | 7.2 | 10.7 |
| 3 mg ABA + 20 mg AVG | 4.8 | 11.7 | 16.0 |
| 30 mg ABA + 20 mg AVG | 7.8 | 23.7 | 32.3 |

The application timing of AVG was also examined with varieties Colossus Formula Mix and Delta Premium Pure White. AVG was applied 24 h prior to, the same time as, or 24 hours after ABA application. Plants not receiving AVG treatments were treated with the same volume of water on the day of the AVG treatment. Therefore, in this experiment the irrigation was stopped at 24 hours after ABA treatment. Results with Colossus Formula Mix (Table 18) showed that AVG application timing did not affect Pansy shelf life. ABA related Pansy yellow leaf number decreased at 9 days after treatment when AVG was applied 24 hours prior to or at the same time as ABA application. Yellow leaf number also decreased when AVG was applied 24 h after ABA treatment.

TABLE 18
Effect of AVG applied at different timing on ABA related Pansy
(Colossus Formula Mix) yellow leaf number and shelf life.

| | | Yellow leaf | |
|---|---|---|---|
| | | number | |
| | | Days after | |
| | | treatment | |
| Treatment | Shelf Life (days) | 3 | 9 |
| Control | 3.7 | 0.7 | 2.3 |
| 30 mg ABA | 6.2 | 9.0 | 13.0 |
| 2 mg AVG at −1 d | 3.5 | 0.5 | 2.2 |
| 30 mg ABA + 2 mg AVG at −1 d | 6.2 | 7.3 | 10.3 |
| 2 mg AVG at 0 d | 3.7 | 0.3 | 1.8 |
| 30 mg ABA + 2 mg AVG at 0 d | 6.3 | 7.0 | 9.8 |
| 2 mg AVG at +1 d | 4.2 | 0.3 | 1.3 |
| 30 mg ABA + 2 mg AVG at +1 d | 6.3 | 8.2 | 11.8 |

AVG at −1 d: AVG was applied 1 day prior to ABA application.
AVG at 0 d: AVG was applied the same as ABA application.
AVG at +1 d: AVG was applied 1 day after ABA application.

The results with variety Delta Premium Pure White were similar (Table 19). Pansy shelf life was not affected whether AVG was applied 24 hours prior to, the same time as or 24 hours after ABA treatment. The ABA related Pansy yellow leaf number was decreased by AVG application at 3 or 9 days after ABA treatment. There was no difference among the three AVG application timings.

TABLE 19
Effect of AVG applied at different timing on ABA related Pansy
(Delta Premium Pure White) yellow leaf number and shelf life.

| | | Yellow leaf | |
|---|---|---|---|
| | | number | |
| | | Days after | |
| | | treatment | |
| Treatment | Shelf Life (days) | 3 | 9 |
| Control | 5.0 | 0.3 | 1.5 |
| 30 mg ABA | 8.7 | 5.2 | 11.0 |
| 2 mg AVG at −1 d | 5.3 | 0.3 | 1.5 |
| 30 mg ABA + 2 mg AVG at −1 d | 8.5 | 3.3 | 7.3 |
| 2 mg AVG at 0 d | 5.2 | 0.0 | 1.7 |
| 30 mg ABA + 2 mg AVG at 0 d | 8.7 | 2.8 | 7.7 |
| 2 mg AVG at +1 d | 5.7 | 0.2 | 1.5 |
| 30 mg ABA + 2 mg AVG at +1 d | 8.5 | 3.5 | 7.7 |

AVG at −1 d: AVG was applied 1 day prior to ABA application.
AVG at 0 d: AVG was applied the same as ABA application.
AVG at +1 d: AVG was applied 1 day after ABA application.

### Example 12

1-Methylcyclopropene (1-MCP; MCP), an ethylene action inhibitor, was also tested for its effect on ABA related Pansy leaf yellowing. Pansy (variety: Colossus Formula Mix) was treated with 0, 3 or 30 mg ABA and then transferred to a closed container for 12 hours. Ethyl-Bloc was placed in a beaker mixed with buffer solution to release MCP inside the closed container to reach a concentration of 10 μL L⁻¹. Plants without MCP treatment were placed in a different closed container for 12 hours with no MCP exposure inside the container.

After MCP treatment, plants were removed from the container and held under no water (drought) conditions. The shelf life for MCP treated plants was not different than for control plants (Table 20). The shelf life for MCP+3 mg ABA combination treatment was not different from the 3 mg ABA alone treatment. The combination of 10 μL L⁻¹ MCP with 30 mg ABA further increased shelf life beyond the 30 mg ABA treatment. Plants treated with the combination of MCP and ABA had a numerically lower yellow leaf number compared to plants treated with same concentration of ABA alone.

TABLE 20
Effect of MCP on ABA related Pansy (Colosus Formula Mix)
yellow leaf number and shelf life

| | | Yellow leaf number | |
|---|---|---|---|
| | | Days after treatment | |
| Treatment | Shelf Life (days) | 3 | 8 |
| Control | 4.5 | 0.5 | 2.5 |
| 3 mg ABA | 5.2 | 4.0 | 6.5 |
| 30 mg ABA | 6.5 | 8.0 | 12.3 |
| 10 μL L⁻¹ MCP | 4.2 | 0.2 | 1.7 |
| 3 mg ABA + 10 μL L⁻¹ MCP | 5.2 | 2.8 | 4.2 |
| 30 mg ABA + 10 μL L⁻¹ | 7.3 | 7.5 | 11.7 |
| MCP | | | |

In a similar study with the Pansy variety Delta Premium Pure White, plants were treated with MCP for 24 hours immediately after ABA treatment. MCP did not affect the shelf life of Pansy plants treated with 3 or 30 mg ABA (Table 21). MCP decreased the yellow leaf number at 3 or 8 days after treatment, respectively.

TABLE 21
Effect of MCP on ABA related Pansy (Delta Premium
Pure White) yellow leaf number and shelf life

| | | Yellow leaf number | |
|---|---|---|---|
| | | Days after treatment | |
| Treatment | Shelf Life (days) | 3 | 8 |
| Control | 7.7 | 0.3 | 0.8 |
| 3 mg ABA | 8.3 | 2.5 | 3.8 |
| 30 mg ABA | 10.2 | 7.2 | 9.0 |
| 10 μL L⁻¹ MCP | 7.0 | 0.0 | 0.7 |
| 3 mg ABA + 10 μL L⁻¹ MCP | 8.0 | 1.5 | 2.5 |
| 30 mg ABA + 10 μL L⁻¹ | 10.3 | 5.8 | 6.0 |
| MCP | | | |

Pansy (Delta Premium Pure White) was also treated with MCP at 24 hours before, 0 or 24 hours after 0, 3 or 30 mg ABA treatment. MCP applied at different times did not affect Pansy shelf life whether treated with 3 mg ABA or 30 mg ABA (Table 22). MCP applied 24 hours prior to, or 0 or 24 hours after ABA treatment reduced the yellow leaf number. Pansy plants had a lower yellow leaf number when MCP was applied 24 hours prior to or 0 h after ABA compared to MCP applied 24 hours after ABA treatment.

TABLE 22
Effect of MCP applied at different timing on ABA related Pansy
(Delta Premium Pure White) yellow leaf number and shelf life

| | Shelf | Yellow leaf number | |
|---|---|---|---|
| | life | Days after treatment | |
| Treatment | (days) | 3 | 7 |
| Control | 4.2 | 2.0 | 4.8 |
| 3 mg ABA | 5.7 | 7.2 | 11.7 |
| 30 mg ABA | 9.2 | 13.5 | 20.2 |
| 10 μL L⁻¹ MCP at −24 hrs | 4.0 | 2.0 | 4.0 |
| 3 mg ABA + 10 μL L⁻¹ MCP at −24 hrs | 5.2 | 4.3 | 7.0 |
| 30 mg ABA + 10 μL L⁻¹ MCP at −24 hrs | 7.7 | 9.2 | 14.5 |
| 10 μL L⁻¹ MCP at 0 hrs | 4.0 | 1.8 | 4.0 |
| 3 mg ABA + 10 μL L⁻¹ MCP at 0 hrs | 5.8 | 3.2 | 8.2 |
| 30 mg ABA + 10 μL L⁻¹ MCP at 0 hrs | 8.8 | 9.0 | 13.8 |
| 10 μL L⁻¹ MCP at +24 hrs | 4.0 | 2.2 | 4.0 |
| 3 mg ABA + 10 μL L⁻¹ MCP at +24 hrs | 5.8 | 5.7 | 9.8 |
| 30 mg ABA + 10 μL L⁻¹ MCP at +24 hrs | 9.0 | 9.8 | 16.7 |

MCP at −24 hrs: Plants were placed in a closed container filled with 10 μL L⁻¹ MCP for 12 hours, beginning 24 hrs prior to ABA application.
MCP at 0 hrs: Plants were placed in a closed container filled with 10 μL L⁻¹ MCP for 12 hours, beginning right after ABA application.
MCP at +24 hrs: Plants were placed in a closed container filled with 10 μL L⁻¹ MCP for 12 hours, beginning 24 hrs after ABA application.

### Example 13

10 μL L⁻¹ MCP, 20 mg AVG, 2 mg BA, or their combinations were applied to Pansy plants with or without 30 mg ABA. Without ABA, the shelf life of Pansy plants ranged from 3.5 to 4.5 days (Table 23). With ABA, the shelf life of Pansy plants ranged from 7.8 to 8.5 days. ABA was the only factor that affected Pansy shelf life. MCP, AVG, BA, or their combinations did not affect Pansy shelf life with or without ABA.

Without ABA, Pansy plants also developed yellow leaves but maintained them at a low level. BA alone and its combination with AVG decreased the yellow leaf number at 3 and 3 or 7 days after treatment respectively. 30 mg ABA dramatically increased Pansy yellow leaf number. MCP, AVG, BA and their combinations dramatically decreased the ABA induced increase in yellow leaf number. However, none of these treatments completely eliminated Pansy leaf yellowing. Among these treatments, the combination of BA with AVG, MCP, and AVG plus MCP reduced the Pansy yellow leaf number more than the other treatments. The combination of BA with AVG, MCP, or both reduced the yellow leaf number more than BA alone.

TABLE 23
Effect of MCP, AVG, BA and their combinations on ABA related
Pansy (Delta Premium Pure White) yellow leaf number and shelf life

| | | Yellow leaf number | |
|---|---|---|---|
| | | Days after treatment | |
| Treatment | Shelf life (days) | 3 | 7 |
| Control | 4.5 | 4.5 | 7.5 |
| 30 mg ABA | 8.2 | 14.3 | 21.3 |
| 10 μL L⁻¹ MCP | 4.5 | 3.2 | 6.7 |
| 2 mg AVG | 3.5 | 2.0 | 6.3 |
| 2 mg BA | 4.0 | 1.3 | 6.2 |
| 10 μL L⁻¹ MCP + 2 mg AVG | 4.0 | 3.0 | 7.2 |
| 10 μL L⁻¹ MCP + 2 mg BA | 3.5 | 2.3 | 7.0 |
| 2 mg AVG + 2 mg BA | 4.3 | 0.8 | 3.8 |
| 10 μL L⁻¹ MCP + 2 mg AVG + | 3.8 | 2.5 | 4.7 |
| 2 mg BA | | | |
| 10 μL L⁻¹ MCP + 30 mg ABA | 8.2 | 11.5 | 19.2 |
| 2 mg AVG + 30 mg ABA | 8.5 | 8.7 | 14.5 |
| 2 mg BA + 30 mg ABA | 8.2 | 6.7 | 9.5 |
| 10 μL L⁻¹ MCP + 2 mg AVG + | 7.8 | 12.3 | 17.0 |
| 30 mg ABA | | | |
| 10 μL L⁻¹ MCP + 2 mg BA + | 8.0 | 5.2 | 9.0 |
| 30 mg ABA | | | |
| 2 mg AVG + 2 mg BA + 30 mg | 8.3 | 4.8 | 8.5 |
| ABA | | | |
| 10 μL L⁻¹ MCP + 2 mg AVG + | 8.3 | 4.3 | 8.2 |
| 2 mg BA + 30 mg ABA | | | |

### Example 14

GA₃ or GA_{4/7} applied at 0.1 mg or 1 mg per plant were evaluated to determine their effect on reducing ABA related Pansy leaf yellowing and increase shelf life. Neither GA₃ nor GA_{4/7} affected Pansy shelf life alone or in combination with ABA (Table 24). However, both GA₃ and GA_{4/7} reduced the Pansy yellow leaf number caused by either 3 mg or 30 mg ABA. GA_{4/7} reduced the number of yellow leaves more than GA₃. GA₃ and GA_{4/7} treatment had no apparent effect on plant elongation.

TABLE 24
Effect of GA3, or GA₄₊₇ on ABA related Pansy (Delta
Premium Pure White) yellow leaf number and shelf life.

| | | Yellow leaf number | |
|---|---|---|---|
| | | Days after treatment | |
| Treatment | Shelf life (days) | 3 | 7 |
| Control | 4.3 | 2.0 | 3.8 |
| 3 mg ABA | 5.2 | 5.3 | 10.5 |
| 30 mg ABA | 7.2 | 12.2 | 18.2 |
| 0.1 mg GA₃ | 4.3 | 1.3 | 3.8 |
| 1 mg GA₃ | 4.0 | 1.2 | 3.7 |
| 0.1 mg GA_{4/7} | 4.3 | 1.5 | 4.0 |
| 1 mg GA_{4/7} | 4.2 | 1.5 | 3.5 |
| 0.1 mg GA₃ + 3 mg ABA | 5.0 | 3.0 | 7.3 |
| 0.1 mg GA₃ + 30 mg ABA | 7.7 | 6.2 | 11.5 |
| 1 mg GA₃ + 3 mg ABA | 5.7 | 2.5 | 6.7 |
| 1 mg GA₃ + 30 mg ABA | 7.0 | 6.5 | 10.5 |
| 0.1 mg GA_{4/7} + 3 mg ABA | 4.8 | 3.0 | 6.7 |
| 0.1 mg GA_{4/7} + 30 mg ABA | 7.8 | 3.7 | 8.0 |
| 1 mg GA_{4/7} + 3 mg ABA | 4.7 | 2.3 | 5.8 |
| 1 mg GA_{4/7} + 30 mg ABA | 7.3 | 4.5 | 6.7 |

1. A method for the treatment of an ABA sensitive plant comprising applying an effective amount of at least one ABA analog to said plant wherein said ABA analog is selected from the group consisting of PBI-524, PBI-429, PBI-696, PBI-702 and PBI-488.

2. A method for the treatment of an ABA sensitive plant comprising applying an effective amount of ABA and an ABA antagonist to said plant.

3. The method of claim 2 wherein the ABA antagonist is PBI-51.

4. A method for the treatment of an ABA sensitive plant comprising applying an effective amount of ABA and a cytokinin to said plant.

5. The method of claim 4 wherein the cytokinin is an adenine-type cytokinin or a phenylurea-type cytokinin.

6. The method of claim 4 wherein the cytokinin is N₁-(2-chloro-4-pyridyl)-N₃-phenylurea or 6-benzylaminopurine.

7. A method for the treatment of an ABA sensitive plant comprising applying an effective amount of ABA and the ethylene biosynthesis inhibitor AVG and/or the ethylene action inhibitor MCP to said plant.

8. The method of claim 7 that further comprises applying BA.

9. A method for the treatment of an ABA sensitive plant comprising applying an effective amount of ABA and gibberellic acid (GA₃) to said plant.

10. A method for the treatment of an ABA sensitive plant comprising applying an effective amount of ABA and gibberellin A₄₊₇ (GA4+7) to said plant.
